(19) United States
(12) Patent Application Publication
(10) Pub. No.: US 2023/0122227 A1
(43) Pub. Date: Apr. 20, 2023

(54) BODY TISSUE MONITOR, BODY TISSUE PRESERVATION SYSTEM AND CORRESPONDING METHOD

(71) Applicant: UNIVERSITY OF NEWCASTLE UPON TYNE, Newcastle upon Tyne (GB)

(72) Inventors: William Earl SCOTT, III, Newcastle upon Tyne (GB); Leo FREITAS, Newcastle upon Tyne (GB)

(21) Appl. No.: 17/913,933

(22) PCT Filed: Mar. 26, 2021
(86) PCT No.: PCT/GB2021/050756

§ 371 Date: Sep. 23, 2022

(30) Foreign Application Priority Data
Mar. 26, 2020 (GB) 2004378.2

(51) Int. Cl. A01N 1/02

(52) U.S. Cl. CPC A01N 1/0289; A01N 1/0247

(57) ABSTRACT

A body tissue preservation system for storage and preservation of body tissue, the system comprising a body tissue monitor to determine a status of the body tissue, wherein the body tissue monitor comprises: at least one sensor configured to obtain sensor data based on a plurality of measurements of the body tissue and/or the environment surrounding the body tissue; and a controller arranged to receive the sensor data from the at least one sensor, wherein the controller is configured to: detect one or more trigger events in the sensor data, wherein each trigger event comprises sensor data which satisfies a first threshold criterion; select, for each of the one or more trigger events, a window of the sensor data associated with the trigger event; identify, for each of the one or more windows, a subset of the sensor data corresponding to the selected window; determine, for each of the one or more selected windows, whether the corresponding identified subset of sensor data satisfies a second threshold criterion; determine a status of the body tissue based on identified subsets which satisfied the second threshold criterion; and provide a status output signal based on the determined status of the body tissue.

### TECHNICAL FIELD

The present disclosure relates to systems and methods for monitoring body tissue. In particular, systems and methods relate to storage and preservation of body tissue, and the monitoring thereof.

### BACKGROUND

In some cases, one or more organs may be harvested from a person after they have died (or voluntarily while still alive), and these organs can be used in another person. In which case, a surgeon may remove a relevant organ from the patient. The organ is then transferred so that it can be inserted into another patient. During this process, there will be a time period in which the organ is not connected to either patient, and this organ is to be maintained in a suitable state so that it may still be useful once it has been inserted into a patient. In this time period the organ may have to be transported, such as from one hospital to another. Storage apparatuses have been disclosed which are designed to facilitate this transfer of an organ.

Aspects of the present disclosure seek to provide improved systems and methods for the storage and/or preservation of body tissue.

### SUMMARY

Aspects of the disclosure are set out in the independent claims and optional features are set out in the dependent claims. Aspects of the disclosure may be provided in conjunction with each other, and features of one aspect may be applied to other aspects.

In an aspect, there is provided a body tissue preservation system for storage and preservation of body tissue. The body tissue preservation system comprises a body tissue monitor for monitoring a body tissue to determine a status of the body tissue. The body tissue monitor comprises: at least one sensor configured to obtain sensor data based on a plurality of measurements of the body tissue and/or the environment surrounding the body tissue; and a controller arranged to receive the sensor data from the at least one sensor. The controller is configured to: detect one or more trigger events in the sensor data, wherein each trigger event comprises sensor data which satisfies a first threshold criterion; select, for each of the one or more trigger events, a window of the sensor data associated with the trigger event; identify, for each of the one or more windows, a subset of the sensor data corresponding to the selected window; determine, for each of the one or more selected windows, whether the corresponding identified subset of sensor data satisfies a second threshold criterion; determine a status of the body tissue based on identified subsets which satisfied the second threshold criterion; and provide a status output signal based on the determined status of the body tissue.

Embodiments may enable more reliable storage and preservation of body tissue. This may find particular utility in the field of organ transplants, where an organ may be stored and/or transported for an extended period of time. Embodiments may facilitate the identifying of organs which may or may not be suitable for transplant based on the properties that organ experienced during storage and/or transport. Embodiments may enable greater reliability in identifying organs as being suitable for implantation after they have been stored/preserved. Examples may enable a viability of the body tissue for transplant to be determined and output by the system.

The system may be configured to store and preserve extracorporeal body tissue, such as body tissue to be stored and/or transported between two locations when located outside of a patient's body. The system may be configured to perfuse body tissue. The system may be configured to persufflate the body tissue. The body tissue preservation system may comprise a body tissue persufflation system. The body tissue may be body tissue for a transplant. The system may be configured to monitor the body tissue during storage and/or transport. The status output signal may provide an indication of the viability of the body tissue for transplant. The system may comprise a container unit arranged to receive the body tissue. The storing, preserving, perfusing and/or persufflating the body tissue may be with the body tissue in the container unit. The container unit may comprise the at least one sensor. Determining a status of the body tissue may comprise determining one or more statuses of the body tissue (e.g. determining a plurality of statuses).

The system may include at least one adjuster operable to adjust a property of the body tissue and/or the environment surrounding the body tissue. For example, the controller, sensor and the adjuster may enable automatic control of one or more operational parameters of the storage and/or preservation of body tissue. The controller may be configured to control operation of the adjuster to adjust the property of the body tissue and/or the environment surrounding the body tissue in the event that it determines that an identified subset of the sensor data satisfies the second threshold criterion. The adjuster may be operable to adjust a property of the persufflation of the body tissue. The controller may be configured to control the adjuster to adjust at least one of: (i) a flow of persufflation fluid to the body tissue, and (ii) a pressure of persufflation fluid flowing through the body tissue.

Selecting a window of the sensor data may comprise selecting a time window of the sensor data. Selecting a window of the sensor data may comprise selecting a window based on one or more other parameters, such as based on the senor (e.g. physical characteristics and/or measurements from the sensor). The system may comprise a plurality of sensors, each configured to obtain sensor data based on a plurality of measurements for the body tissue and/or the environment surrounding the body tissue. The controller may be configured to determine whether an identified subset of data from a first sensor satisfies the second threshold criterion based also on data from a second sensor. The controller may be configured to determine whether an identified subset of data from the first and/or second sensor satisfies a plurality of threshold criteria.

In the event that the controller detects a trigger event in sensor data from the first sensor, the controller may be configured to select a window associated with the trigger event in both the sensor data from the first sensor and the sensor data from the second sensor. The controller may be configured to detect a trigger event in the event that both: (i) a first measurement from a first sensor satisfies a first partial threshold criterion, and (ii) a second measurement from a second sensor satisfies a second partial threshold criterion. For example, partial threshold criteria may enable a threshold criterion only to be satisfied if its constituent partial threshold criteria are satisfied, e.g. a trigger event may only be detected (a first threshold criterion satisfied) in the event that two separate partial threshold criteria are satisfied, such as where the two partial threshold criteria relate to data from different sensors.

The controller may be configured to detect that sensor data satisfies the first threshold criterion in the event that at least one of: (i) the sensor data is outside a selected range, (ii) a change in the sensor data is above a threshold amount, and (iii) a time threshold has been reached. The controller may be configured to determine that an identified subset of the sensor data satisfies a second threshold criterion in the event that at least one of: (i) the sensor data is outside a selected range for a threshold period of time within the selected time window, (ii) the sensor data changes more than a threshold amount within the selected time window, and (iii) one or more selected patterns are identified in the sensor data within the selected time window. An identified subset of the sensor data may comprise data from a plurality of sensors. Determining that the sensor data for the identified subset satisfies a second threshold criterion may comprise determining that: (i) data from a first sensor satisfies the second threshold criterion, (ii) data from a second sensor satisfies the second threshold criterion, (iii) a cross-correlation between the data from the first and second sensors satisfies the second threshold criterion. The controller may be configured to output an alert in the event that it is determined that a corresponding identified subset of the sensor data satisfies the second threshold criterion.

The status output signal may include each identified subset of the sensor data which satisfied the second threshold criterion. The status output signal may include the identified subsets of the sensor data. The status output signal may include the sensor data. The status output signal may include data for each of the one or more trigger events. The status output signal may provide an ordered series of notable events in the sensor data. The notable events may include at least one of: (i) the trigger events, (ii) the identified subsets, and (iii) the identified subsets which satisfied the second threshold criterion. The controller may be configured to provide the status output signal comprising data links arranged to enable a user to select a notable event and to see the sensor data associated with that notable event.

The controller may be configured to: select, for each of the one or more identified subsets which satisfied the second threshold criterion, a second window of the sensor data, wherein the second window is associated with the window of the identified subset; identify, for each of the one or more selected second windows, a subset of the sensor data corresponding to each second window; determine, for each of the one or more selected second windows, whether the corresponding identified subset of the sensor data satisfies a third threshold criterion; and determine the status of the body tissue based on the identified subsets which satisfied the third threshold criterion. The controller may be configured to determine the status of the body tissue by assigning weightings to subsets of the sensor data for the body tissue. Each weighting may be selected based on associated threshold criterions that the corresponding subset of sensor data satisfied.

In an aspect, there is provided a body tissue monitor for monitoring a body tissue to determine a status of the body tissue. The body tissue monitor comprises: at least one sensor configured to obtain sensor data based on a plurality of measurements of the body tissue and/or the environment surrounding the body tissue; and a controller arranged to receive the sensor data from the at least one sensor, wherein the controller is configured to: detect one or more trigger events in the sensor data, wherein each trigger event comprises sensor data which satisfies a first threshold criterion; select, for each of the one or more trigger events, a time window of the sensor data associated with the trigger event; identify, for each of the one or more time windows, a subset of the sensor data corresponding to the selected window; determine, for each of the one or more selected time windows, whether the corresponding identified subset of sensor data satisfies a second threshold criterion; determine a status of the body tissue based on identified subsets which satisfied the second threshold criterion; and provide a status output signal based on the determined status of the body tissue. Methods may comprise correlating windows of events in the sensor data (e.g. where sensor data satisfies a first threshold criterion), such as to identify activities (e.g. patterns at higher layers of abstraction) which satisfy a selected threshold criterion. This may be based on a cross-correlation between data from multiple sensors.

In an aspect, there is provided a method of storing and preserving body tissue. The method comprises: obtaining sensor data defining a stream of measurements for the body tissue and/or the environment surrounding that body tissue; detecting one or more trigger events in the sensor data, wherein each trigger event comprises sensor data which satisfies a first threshold criterion; selecting, for each of the one or more trigger events, a window of the sensor data associated with the trigger event; identifying, for each of the one or more windows, a subset of the sensor data corresponding to the selected window; determining, for each of the one or more selected windows, whether the corresponding identified subset of the sensor data satisfies a second threshold criterion; determining a status of the body tissue based on identified subsets which satisfied the second threshold criterion; and providing a status output signal based on the determined status of the body tissue.

Aspects of the present disclosure may provide systems and methods for monitoring body tissue to determine a status of the body tissue. Such aspects may include any of the sensor data processing steps disclosed herein.

Aspects of the present disclosure may provide one or more computer program products comprising computer program instructions configured to program a controller to perform any of the methods disclosed herein.

### FIGURES

Some examples of the present disclosure will now be described, by way of example only, with reference to the figures, in which:

FIG. 1 is a schematic diagram of an example body tissue preservation system.

FIGS. 2a to 2d show an exemplary series of Pressure versus Time graphs.

FIGS. 3a to 3d show an exemplary series of Pressure and Acceleration versus Time graphs.

In the drawings like reference numerals are used to indicate like elements.

### SPECIFIC DESCRIPTION

The present disclosure relates to monitoring of a body tissue preservation system. The body tissue preservation system includes a body tissue monitor which is configured to monitor body tissue to determine a status of the body tissue. The body tissue monitor is configured to monitor sensor data for the body tissue (and/or its surrounding environment). The sensor data includes a plurality of measurements for the body tissue. Monitoring the sensor data includes identifying measurement values which may indicate that the status of the body tissue has been influenced, such as measurement values lying outside an expected range. These measurement values, and other measurement values occurring close to them (e.g. in time), are then further analysed to identify one or more patterns which may be apparent when considering the measurement values on a larger scale. The status of the body tissue may be determined based on any such identified patterns.

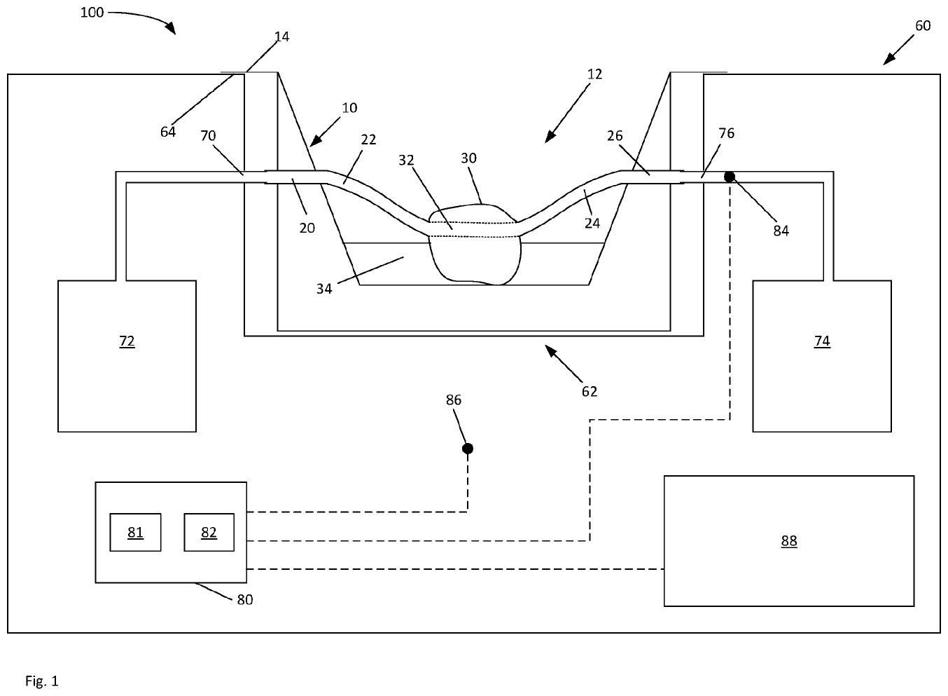

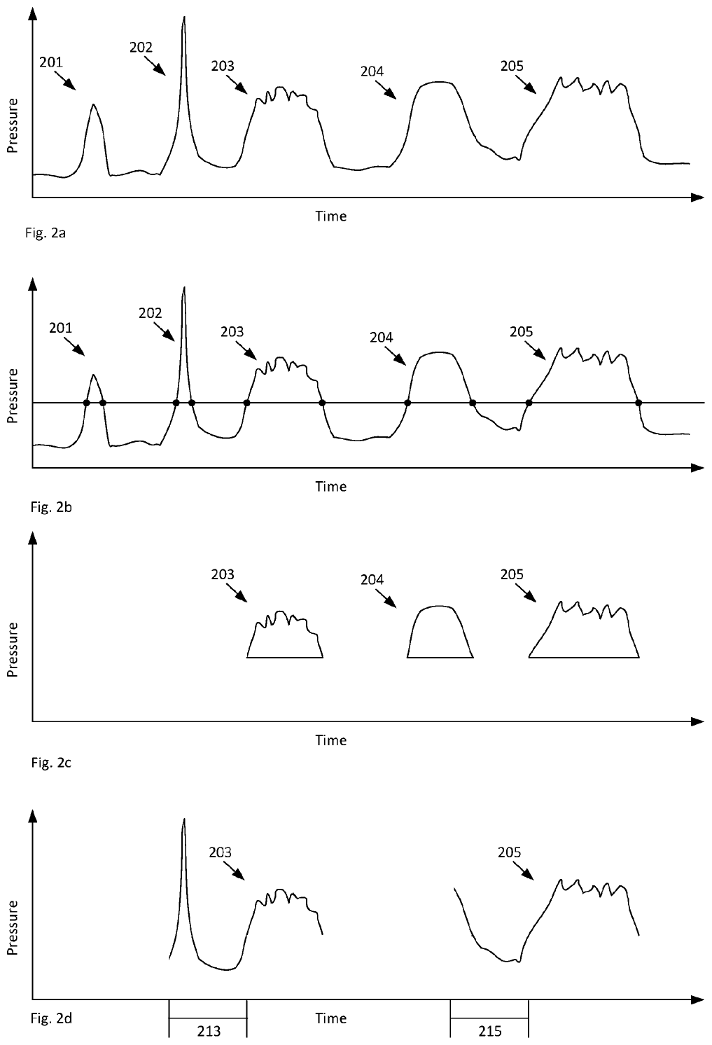

One specific example of a body tissue preservation system and a method of using such will now be described with reference to FIGS. 1 to 2d. It will be appreciated in the context of the present disclosure that this is one specific example, and that it is not to be considered limiting. Numerous alternatives of this system are described later to show that not all of the features of FIGS. 1 to 2d are required and/or that additional features may also be included.

FIG. 1 shows an example body tissue preservation system 100. The body tissue preservation system is made up of two parts: a container unit 10 and a base unit 60. FIG. 1 shows an assembled body tissue preservation system 100, with the container unit 10 received in the base unit 60.

The container unit 10 includes a body tissue receiving portion 12 and a flange 14. The container unit 10 also includes an inlet 20, an outlet 26 and one or more channels connected to the inlet 20 or outlet 26. A first channel 22 is connected to the inlet 20 to define a flow path from external to the container unit 10 into the body tissue receiving portion 12. A second channel 24 is connected to the outlet 26 to define a flow path from the body tissue receiving portion 12 to outside the container unit 10. One or more walls of the container unit 10 define a recess which provides the body tissue receiving portion 12. The inlet 20 and outlet 26 each extend through a portion of a wall defining the body tissue receiving portion 12. The flange 14 extends radially outward from the container unit 10. As shown in FIG. 1 a body tissue 30 having at least one lumen 32 is stored in the container unit 10 (in the body tissue receiving portion 12). The body tissue 30 is resting on a bottom surface of the body tissue receiving portion 12 of the container unit 10, and the body tissue 30 is at least partially immersed in a preservation fluid 34.

The container unit 10 is configured as an insert for a base unit 60. It is sized and shaped to fit within a corresponding recess in a base unit 60. The container unit 10 is arranged to house a body tissue 30 to be stored. This includes being configured to store the body tissue 30 in a pool of preservation fluid 34. The container unit 10 is configured as a single-use component which is to be disposed of after having received a body tissue 30 for storage and preservation.

The body tissue receiving portion 12 is sized and shaped to receive a body tissue 30 to be stored and/or preserved. The body tissue receiving portion 12 is configured to store a pool of preservation fluid 34 for the body tissue 30 to be stored. The body tissue receiving portion 12 is configured to receive a body tissue 30, which rests on the lower surface of the body tissue receiving portion 12. Body tissue 30 resting on the lower surface will be immersed in the pool of preservation fluid 34 retained in the body tissue receiving portion 12.

The flange 14 is configured to support the container unit 10 when inserted into a base unit 60. The flange 14 is arranged to enable the container unit 10 to be inserted into the base unit 60 and retained in position within the base unit 60. The flange 14 provides a lip from which the container unit 10 may be suspended into the base unit 60.

The inlet 20 is arranged to receive a source of incoming fluid from the base unit 60. The inlet 20 is configured to provide a flow path to enable fluid external to the container unit 10 to be delivered into the body tissue receiving portion 12. The first channel 22 is connected to the inlet 20 to extend the fluid flow path into the body tissue receiving portion 12. The first channel 22 is arranged to be inserted into the lumen 32 of the body tissue 30 stored in the body tissue receiving portion 12 (the lumen 32 may be a vein or an artery of an organ). The first channel 22 is configured to connect a source of fluid external to the container unit 10 (in the base unit 60) to the body tissue 30 stored in the container unit 10. The first channel 22 is arranged to enable delivery of fluid into the body tissue 30 stored in the container unit 10.

The outlet 26 is arranged to enable fluid to leave the body tissue receiving region. The outlet 26 is arranged to enable fluid to flow out from the body tissue receiving region into a corresponding region of the base unit 60. The second channel 24 is connected to the inlet 20 and is also arranged to be inserted into a lumen 32 of body tissue 30 stored in the body tissue receiving portion 12. The second channel 24 and outlet 26 provide a fluid flow path for fluid in the lumen 32 of the body tissue 30 to be delivered out of the body tissue receiving portion 12. This fluid includes fluid pumped into the body tissue 30 through the inlet 20 and first channel 22. The first and second channels are arranged to enable circulation of fluid so that fluid may be delivered to the body tissue 30 through the inlet 20 and first channel 22 and then out of the body tissue 30 and away from the container unit 10 through the second channel 24 and the outlet 26.

The base unit 60 includes a container unit receiving portion 62, a connection surface 64, a controller 80 and a display screen 88. The controller 80 includes a processor 81 and a data store 82. The base unit 60 also includes a first fluid store 72, a fluid outlet 70, a fluid inlet 76 and a second fluid store 74. The base unit 60 also includes a first sensor 84 and a second sensor 86.

The first fluid store 72 is connected to the fluid outlet 70. The fluid outlet 70 provides a connection into the container unit receiving portion 62 of the base unit 60 from a body of the base unit 60. The second fluid store 74 is connected to the fluid inlet 76. The fluid inlet 76 provides a connection from the container unit receiving portion 62 into a body of the base unit 60. The first sensor 84 is provided between the fluid inlet 76 and the second fluid store 74. The second sensor 86 is provided in a body of the base unit 60. The connection surface 64 is a top surface of the base unit 60. The container unit receiving portion 62 is a recess provided in the top surface of the base unit 60. The controller 80 is connected to each of the first sensor 84, the second sensor 86, and the display screen 88.

The base unit is configured for storing and preserving body tissue 30. Body tissue 30 in the container unit 10 may be stored and preserved inside the base unit 60. The base unit 60 is configured to control conditions within the environment of the container unit 10, as well as to control the supply of one or more preserving fluids to the body tissue 30 in the container unit 10. The base unit 60 is configured to be portable. Although not shown, a lid and handle are provided to facilitate transport of the base unit 60. The base unit 60 is configured to receive a container unit 10 containing a body tissue 30 to be stored and/or preserved and to enable connection of components of the base unit 60 to the container unit 10.

The container unit receiving portion 62 is arranged to receive a container unit 10. The container unit receiving portion 62 is arranged to hold a container unit 10 in the container unit receiving portion 62 to enable components of the base unit 60 to be connected to the container unit 10. The container unit receiving portion 62 is configured to support the container unit 10 to enable transport of the container unit 10 in the base unit 60 (while inhibiting damage to any body tissue 30 carried in the container unit 10). The container unit receiving portion 62 is sized and shaped to receive a container unit 10 inside, and to permit a snug fit of the container unit 10.

The top surface of the base unit 60 is arranged support a flange 14 of a container unit 10 inserted in the base unit 60, and to enable the container unit 10 to be received in the container unit receiving portion 62. The top surface provides a surface against which the flange 14 of a container unit 10 may abut to secure the container unit 10 in the container unit receiving portion 62 of the base unit 60.

The first fluid store 72 comprises a source of preservation fluid to be delivered to the body tissue 30 in the container unit 10. The first fluid store 72 is a tank of preservation fluid. The first fluid store 72 is provided in a body of the base unit 60. Fluid is stored under pressure in the fluid store.

The fluid outlet 70 is arranged to be connectable to a fluid inlet 20 of a container unit 10 in the container unit receiving portion 62. The fluid outlet 70 is arranged to enable fluid from a fluid source in the base unit 60 to be delivered into the container unit 10 (and to body tissue 30 contained in the container unit 10). The fluid outlet 70 may connect to the fluid inlet 20 of the container unit 10 to provide a flow path from the first fluid store 72 of the base unit 60 into the body tissue 30 in the container unit 10.

The fluid inlet 76 is arranged to be connectable to fluid in the container unit receiving portion 62. The fluid inlet 76 is arranged to be connectable to a fluid outlet 26 of a container unit 10 in the container unit receiving portion 62. The fluid inlet 76 is configured to enable fluid to pass out from the container unit 10 and into the body of the base unit 60. The fluid inlet 76 is connectable to the fluid outlet 26 of the container unit 10 to enable fluid to flow from within body tissue 30 in the container unit 10 in to the second fluid store 74 in the base unit 60. The second fluid storage tank is arranged to receive fluid from the fluid inlet 76 for storage. The arrangement may enable preserving fluid from the first fluid store 72 to be delivered to the body tissue 30 in the container unit 10, and for used fluid which has passed through the body tissue 30 to be delivered to the second fluid storage tank.

The display screen 88 is configured to display one or more output values from the system 100. The display screen 88 is configured to output sensor measurement values. The display screen 88 is configured to provide an output to facilitate user input, such as to enable a user to use the display screen 88 to input data for controlling the storage and/or preservation of body tissue 30 in the container unit 10.

The first sensor 84 is arranged to provide including measurement values for at least one property of fluid flow. In this example, the sensor is arranged to provide an indication of a pressure of the preservation fluid after it has been delivered to the body tissue 30 in the container unit 10. In this example, the sensor is a pressure sensor configured to obtain an indication of a pressure of fluid which has been delivered to the container unit 10.

The second sensor 86 is arranged to provided measurement values for a different property to the first fluid. In this example, the second sensor 86 is arranged in the body of the base unit 60, and is configured to obtain a measurement of a property of the preservation system 100. In this example, the second sensor 86 is configured to provide an indication of one or more movement properties of the system 100. The second sensor 86 in this example is an accelerometer.

The two sensors provide sensor data comprising a plurality of measurement values. In this example, each sensor provides a time-ordered series of measurement values. The measurement values are taken over time to provide a stream of data, such as continuous measurement data (e.g. measurement data obtained on an at least semi-regular basis).

The controller 80 is configured to receive sensor data from the sensors and to process the sensor data to determine a status of the body tissue 30. The data store 82 stores instructions for processing the sensor data, and the processor 81 is configured to run those instructions to process the sensor data. The instructions comprise a plurality of processing steps for analysing and processing the data. The instructions enable the data to be processed using methods disclosed herein. In this example, the instructions include a series of processing steps which define one or more algorithmic approaches to process the sensor data. The processing steps may include a number of different ways to process data (e.g. to follow a plurality of different discrete or not discrete processing steps), such as to enable the data to be processed in a number of different ways, and also to enable this processing to be traceable and verifiable. The controller 80 is configured to process the data as described below with reference to FIGS. 2a to 2d. Based on this processing of data, the controller 80 is configured to determine a status of the body tissue 30 and to provide an output based on this status of the body tissue 30. The output may be provided both during storage and/or transport, and/or after storage and/or transport (e.g. prior to implanting the body tissue in a patient). The controller 80 is configured to control the output provided to the display screen 88.

When assembled, the container unit 10 is inserted into the container unit receiving portion 62 of the base unit 60. The flange 14 of the container unit 10 is resting on the connection surface 64 of the base unit 60. The outlet 70 of the base unit 60 is connected to the inlet 20 of the container unit 10, and the outlet 26 of the container unit 10 is connected to the inlet 76 of the base unit 60. The first and second channels of the container unit 10 are connected to the lumen 32 of the body tissue 30. A fluid flow path is defined from the first fluid store 72 through the outlet 70 of the base unit 60 into the inlet 20 of the container unit 10, through the first channel 22 and into the lumen 32 of the body tissue 30, out into the second channel 24, through the outlet 26 of the container unit 10 into the inlet 76 of the base unit 60, and into the second fluid store 74.

Operation of the body tissue preservation will now be described with reference to FIGS. 2a to 2d. In particular, FIG. 2a will be described to show the processing steps performed by the controller 80 for processing the obtained sensor data to determine a status of the body tissue 30.

FIGS. 2a to 2d each show a graph of Pressure versus Time. The pressure values are those obtained from the first sensor 84, and this data is shown as a continuous curve. In this example, the pressure values were obtained for the fluid pressure of fluid being delivered to the body tissue 30 while that body tissue 30 was stored extracorporeally.

FIG. 2a shows a first Pressure versus Time graph. As can be seen, a general base line of the pressure value exists over time, but there are five clearly defined regions where the pressure value deviates from this base line. These deviations are clear, but each deviation has unique or different properties.

A first pressure region 201 has a short and sharp deviation, where the pressure rises steeply, before returning to the base line value. A second pressure region 202 has a shorter sharper deviation. The second pressure region 202 registers the highest pressure value, and as can be seen, the pressure in the second pressure region 202 rises and falls rapidly. A third pressure region 203 includes a more sustained pressure rise, where the pressure remains above the base line value for a longer period of time. During this time period, the pressure is not constant. There are a few oscillations in pressure and/or sharp increases/decreases in pressure value at an elevated pressure value. In the third pressure region 202 there are a plurality of gradient changes while the pressure remains elevated. A fourth pressure region 204 includes a sustained rise in pressure for a longer period of time. In the fourth pressure region 204, the pressure, once elevated remains at a constant pressure before decreasing again. The pressure curve in the fourth pressure region 204 is relatively smooth. A fifth pressure region 205 includes a similar rise to that in the third pressure region 202. Although the rise and fall of the pressure at start and finish of the fifth pressure region 205 is at different rates to that of the third pressure region 202, the fifth pressure region 205 does still exhibit a number of gradient changes while at elevated pressure.

To process this pressure data, the controller 80 is configured to identify regions of interest, and to examine these regions of interest. The stored instructions define how this analysis is to be performed. The instructions include processing of the data at a higher level of abstraction than to just look at instantaneous values. The different levels of abstraction may be selected based on what analysis of the data is being performed (e.g. what threshold is to be satisfied). The level of abstraction may be selected depending on which threshold criterion is being applied. These may depend on the data to be analysed. In this example, the different levels of abstraction are based on time, so that processing data at each subsequent level of abstraction comprises processing data over a longer time period. The threshold criterion may be selected based on the time period over which they are being assessed. However, it is to be appreciated in the context of the present disclosure that the levels of abstraction may use different variables than time (e.g. abstracting over another parameter than time). Larger scale trends are observed which may enable an improved determination of the status of the body tissue 30. Processing the data may include filtering of the data to enable key points/regions of interest to be identified, as well as other suitable processing steps such as agglomeration and/or correlation of the data.

As a first step, FIG. 2b shows an annotated version of the graph of FIG. 1. To process the sensor data, one or more trigger events are identified. Trigger events are identified by detecting an indication that the sensor data satisfies a first threshold criterion. A trigger event includes an indication of an abnormality in the data. The abnormality includes some deviance from an expected measurement value and/or a measurement value no longer falling within a selected range for measurement values. In this example, the first threshold criterion is judged based on a single measurement value. This measurement value is compared to a threshold value to detect the trigger event. In this example, the threshold criterion involves an assessment of whether or not the pressure value exceeds a pressure threshold value.

FIG. 2b shows the same five pressure regions of FIG. 2a. For each region, there are one or more pressure measurements which exceed the pressure threshold value. Ten black circles the intersections between the pressure value and the pressure threshold value. These provide five pairs of circles, where each pair of circles defines the borders of a subset of the data where the pressure is above the pressure threshold value, and where the first threshold criterion is satisfied.

Processing the sensor data includes selecting a window in the sensor data. The window in the sensor data is selected to be a window which takes into account more measurements than those used for the detection of the first trigger event. In this example, selecting a window in the sensor data includes selecting a time window during which measurement values will be used for further analysis.

In this example, there are five trigger events identified. Each of these trigger events is when the pressure first increases above the pressure threshold value. That is, the first, third, fifth, seventh and ninth black circles shown in FIG. 2b illustrate the trigger events. For each of these trigger events a window in the sensor data is selected. Each window is selected to encompass more data than just the measurement which exceeded the pressure threshold value. In this example, each window is selected to encompass all subsequent measurements before the pressure returns below the pressure threshold value. That is, five windows are defined, which correspond to the five pairs of black circles shown. For each of these windows, the controller 80 is configured to obtain sensor data. That is, the controller 80 obtains a subset of the sensor data which corresponds to the window. The subset of data obtained for each window includes the measurement values from the pressure during that time window.

The controller 80 then processes the data for each window. The data for the window is assessed at a macro level, in the sense that any determination based on this data is not just performed for instantaneous pressure values, but also for trends within the window as a whole (e.g. global pressure values within the window, length of overpressure excursion, height of over pressure peak over time, area under the curve, overpressure etc.). The data for each window is then processed to determine if it satisfies a second threshold criterion. Whether the second threshold criterion is satisfied is assessed based on more than just the data corresponding to the trigger event. In this example, the assessment is performed on the data in each window as a whole.

In this example, comparing the data in each window to a second threshold criterion comprises assessing whether or not the data in the window, as a whole, satisfies the second threshold criterion. In this example, satisfying the second threshold is based on the extent to which the body tissue 30 is subject to pressures above the pressure threshold value. This includes an indication of both: (i) absolute values for the pressure to which the body tissue 30 has been subjected, and (ii) the duration of time for which the body tissue 30 has been subjected to the high pressures. In this regard, the second threshold is assessed based on the area under the curve in the window (such as the area under the curve above the threshold value). Determining whether a selected window satisfies the second threshold criterion is assessed based on a total sum of pressure data within the selected window and/or an integral of the pressure curve in the selected window.

In this example, in the event that the total calculated pressure exposure in the window is above a threshold value, it is determined that the window satisfies the second threshold criterion.

FIG. 2c shows the Pressure versus Time graph of FIGS. 2a and 2b except that in FIG. 2c there are only three windows shown, which correspond to the third, fourth and fifth pressure regions. In this example, the other pressure regions of the graph have effectively been filtered (they are not shown). It is to be appreciated in the context of the present disclosure that the other data in the graph is not discarded, but rather to help illustrate the processing steps described herein the graph is focused on only windows corresponding to the third, fourth and fifth pressure regions.

The windows corresponding to the third, fourth and fifth pressure regions are retained at this stage as these are the windows for which the area under the curve is above a threshold value (those are the windows which satisfied the second threshold criterion). As can be seen in FIGS. 2a to 2c, these windows have a larger area under the curve. Even though the second pressure region 202 has the highest pressure values, the area under this curve is not as much, and so the window corresponding to the second pressure region 202 does not satisfy the second threshold criterion.

The status of the body tissue 30 is determined based on these three windows. In this example, it is determined that the significance of an impact caused by a high pressure region is more dependent on the total extent of the impact from that high pressure region than the peak pressure within that high pressure region. Determining the status of the body tissue 30 is based more on the total extent of the impact from that high pressure region than the peak instantaneous pressure values. In this example, determining the status of the body tissue 30 is based on the number of windows which satisfied the second threshold criterion. In this example, determining the status of the body tissue 30 is also based on total exposed pressure within the window (e.g. the area under the curve). The number of windows satisfying the second threshold criterion and the total exposed pressure for those windows is combined to provide a pressure score. The status of the body is determined based on the pressure score.

The pressure score provides an indication of both the amount of sustained high pressure regions and the extent of the pressure within those high pressure regions. The pressure score may be a numeric value using which the status of the body tissue may be determined (e.g. there may be a known mapping between values for the pressure score and status of the body tissue, such as a known amount of deterioration arising from the body tissue being subjected to that pressure score). The magnitude of the score provides an indication of the status of the body tissue 30. For a score indicative of few (or no) high pressure regions, the status of the body tissue 30 will be good. For a score indicative of lots of high pressure regions, where those high pressure regions have a high extent of pressure, the status of the body tissue 30 will be bad. An indication of body tissue 30 viability is provided based on the score, e.g. to suggest whether or not the body tissue 30 is likely to be viable for transplant after it has been stored and preserved in the preservation system 100.

An extension of this process will now be described with reference to FIG. 2d. Instead of determining the status of the body tissue 30 as described with reference to FIG. 2c, an additional step may be provided. FIG. 2d shows the Pressure versus Time graph shown in FIGS. 2a to 2c with some additional regions unfiltered as compared to FIG. 2c. Again, the removal/inclusion of different pressure regions within the graph is for illustrative reasons rather to represent this data actually being discarded/used.

As compared to FIG. 2c, the process of FIG. 2d includes an additional assessment to determine whether or not the windows satisfy the second threshold criterion. In this example, in addition to the assessment described above, to satisfy the second threshold criterion, a window must also conform to certain requirements for the consistency of its measurement (e.g. the shape of the curve in the window). In this example, for a window to satisfy the second threshold criterion it must include a threshold number of turning points (e.g. where the gradient changes between positive and negative/includes a stationary point of inflection). As can be seen, only the windows corresponding to the third and fifth pressure regions satisfy this requirement, and so only these regions satisfy the second threshold criterion.

In this example, for determining the status of the body tissue 30, an additional windowing step is provided, as is use of a third threshold criterion. This is illustrated in FIG. 2d.

In the additional windowing step, a window extension is provided to each of the windows which satisfied the second threshold criterion (those corresponding to the third and fifth pressure regions). The window extension extends the window to include more data than used in FIG. 2c. In this example, the window extension extends the window to include a selected amount of data immediately prior to the window. As shown in FIG. 2d, a third window extension 213 is provided for the window corresponding to the third pressure region 202, and a fifth window extension 215 is provided for the window corresponding to the fifth pressure region 205. Each window extension includes sensor data for a selected time period prior to the relevant window as previously selected.

It is then determined if the data in the extended windows satisfies a third threshold criterion. In this example, the third threshold criterion is configured to examine a relationship between the measurement data in the previously-selected regions and measurement data occurring immediately prior to those previously-selected regions. In this example, determining whether the third threshold criterion is satisfied comprises determining whether or not a short, sharp peak occurred immediately prior to the previously-selected window. In the event that it is determined that there was at least one peak in the window extension (e.g. where the gradient increases and decreases, and if this peak is above a selected threshold, such as 120% the value of the peak pressure in the previously-selected pressure region). It may be determined that if a sharp peak occurs prior to an extended peak with multiple gradient changes, then the third threshold criterion is satisfied. In which case the status of the body tissue 30 is determined (e.g. as described above) based on the extended windows satisfying the third threshold criterion.

With reference to the above-described processing steps, it may be seen that the sensor data is processed in such a way to reveal additional patterns based on which the status of the body tissue 30 may be determined. The amount of data based on which a status of the body tissue 30 is to be determined may be reduced, such as to focus on certain regions of the data more than others. The above-described processing steps may provide more sophisticated and accurate data analysis for determining the status of body tissue 30.

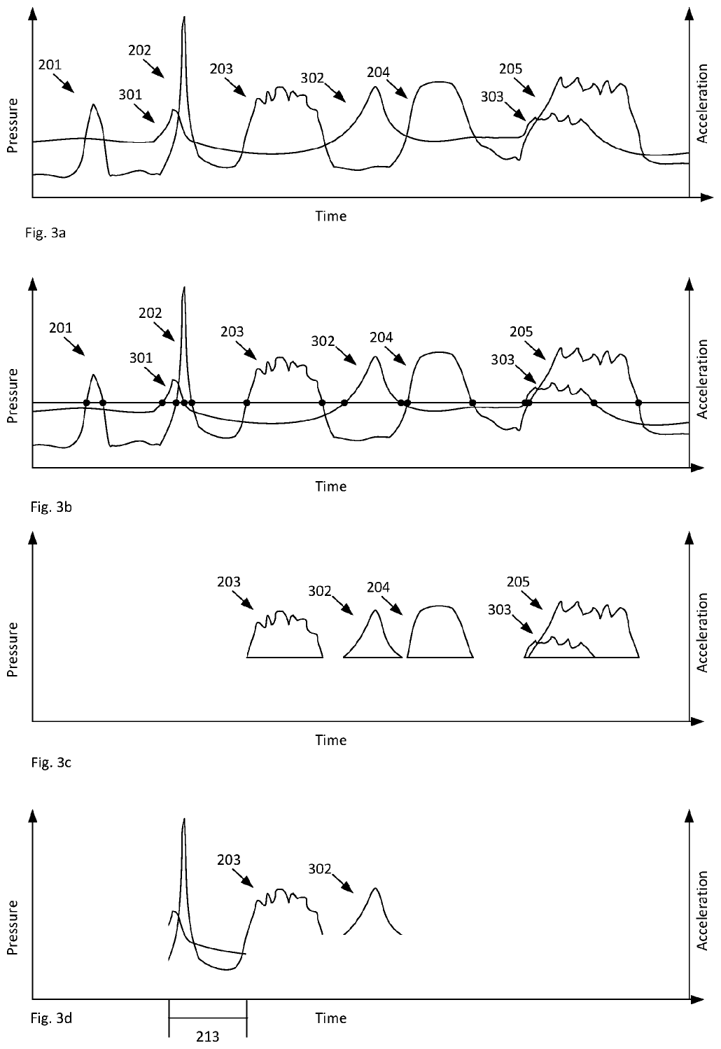

Another example of monitoring sensor data for the body tissue 30 and/or its surrounding environment will now be described with reference to FIGS. 3a to 3d.

FIG. 3a shows a Pressure and Acceleration versus Time graph. In FIG. 3a, the sensor data includes both pressure data and acceleration data. The pressure data is measured by the pressure sensor, and the acceleration data is measured by the accelerometer. The pressure readings are the same as those in FIG. 2a, and so will not be described again. The acceleration data is generally at a baseline value over time (it generally lies within a narrow band of acceleration values). There are three regions in the acceleration data where the measurement values differ significantly from the baseline value.

In a first acceleration region 301, there is an increase in acceleration. This increase occurs for a relatively short time period, and the peak acceleration is not that high. The first acceleration region 301 occurs shortly before the second pressure region 202. There is some overlap between the two, so that the second pressure region 202 begins before the first acceleration region 301 has ended. In a second acceleration region 302, the acceleration increases for a longer period of time, and reaches a higher acceleration peak. The second acceleration is located between the third pressure region 202 and the fourth pressure region 204. In a third acceleration region 303, there is a sustained increase in acceleration. The acceleration in this region is not particularly high, and its gradient changes a lot in this region. The acceleration oscillates somewhat, and/or the acceleration values both increase and decrease while at the elevated acceleration level.

FIG. 3b shows a similar graph to that of FIG. 2b, but including the acceleration data, and with black circles to illustrate when the acceleration data exceeds a threshold value. It is to be appreciated that the threshold acceleration value will be different to the threshold pressure value, but they are just shown as one line on the graph for ease of illustration. As can be seen, there are three windows of acceleration data which are above the threshold value. These windows correspond to the first, second and third acceleration regions. The windows corresponding to the first and third acceleration regions at least partially overlap with the windows corresponding to the second and fifth pressure regions respectively.

In this example, as described above for FIGS. 2a to 2d, a trigger event is defined as an event where the acceleration passes above the acceleration threshold value, and the window of the data which is to be analysed is for the time while the acceleration remains above the acceleration threshold value. Again, the extent of acceleration on the body tissue 30 is measured, and this is based on the area under the curve in the window. Based on this processing, the pressure windows remaining are those discussed previously, and the acceleration windows remaining are those corresponding to the second and third acceleration regions.

FIG. 3c shows these windows, with the remaining regions of the pressure and acceleration data not included. Again, it is to be appreciated that this data is not discarded, but is just not shown to help illustrating the processing steps.

In addition to the processing steps described with reference to FIGS. 2a to 2d, in FIGS. 3a to 3d, processing steps include analysis of the acceleration data and analyse of one data set based also on the other data set. This includes analysing the pressure data based also on the acceleration data, and/or analysing the acceleration data based also on the pressure data.

In FIG. 3c, there are five windows shown which satisfied the second threshold criterion (which had a sufficiently large area under the curve). These are the windows corresponding to the third, fourth and fifth pressure regions, and the windows corresponding to the second and third acceleration regions. When determining the status of the body tissue 30, a cross-correlation is performed between the pressure data and the acceleration data for these windows.

In this example, the cross-correlation is performed between identified windows with overlap. In other words, cross-correlation may not be performed for windows without any overlap. The windows corresponding to the third pressure region 202, the second acceleration region 302 and the fourth pressure region 204 do not have any overlap with another window. That is, in the region of those windows, the other sensor data in that region did not satisfy a first threshold criterion. However, for the window corresponding to the third acceleration region 303 and the fifth pressure region 205, there is an overlap between these windows. The data in these two windows is then compared to assess whether there is a correlation between the two. It is to be appreciated in the context of the present disclosure that a plurality of different trigger points and/or cross-correlations may be used (and analysis of combinations of the different data sets), and that those shown in this example are not to be considered limiting.

Assessing the correlation between the two subsets of the sensor data comprises identifying whether the two windows of data have one or more properties in common. In this example, this assessment is performed by comparing the gradients within that region. The number of gradient shifts (between positive and negative, or stationary inflection) is counted, as is the pattern of these shifts (frequency of occurrence, property of gradient before and after shift, percentage of window between each shift). Based on a comparison between these two properties, it is determined whether or not they are correlated in some way. In this example, this correlation may provide an indication of selected windows in pressure data where the pressure response in that region was caused by the acceleration of system 100. In other words, where the pressure data in that region is not indicative of a property of the body tissue 30 as much as it is a property of the transport of that body tissue 30.

In this example, to determine the status of the body tissue 30, pairs of windows are identified which are determined to have a correlation above a threshold value. When determining the status of the body tissue 30, these windows are not considered as windows which satisfied the second threshold criterion. The status of the body tissue 30 is then determined on the basis of the remaining windows deemed to have satisfied the second threshold criterion. In this example, this will be the windows corresponding to the third pressure region 202, the second acceleration region 302 and the fourth pressure region 204. Determining the status of the body tissue 30 is performed as described above for FIG. 2c. A corresponding or alternative assessment may be used to account for acceleration data. In this example, the status of the body tissue 30 is determined based on the extent of pressure and acceleration on the body tissue 30 (it is based on a sum of area under the curve for the windows which satisfied their second threshold criterion.

A further example is now described with reference to FIG. 3d. As with FIG. 2d, in FIG. 3d additional processing of the pressure data is performed to identify the pressure windows which include a threshold number of turning points. No corresponding assessment is performed for the acceleration windows. As shown in FIG. 3c, the window corresponding to the fifth pressure region 205 has been deemed not to satisfy its second threshold criterion due to its correlation with the window corresponding to the third acceleration region 303. The only windows consider in FIG. 3d are the ones corresponding to the third pressure region 202 and the second acceleration region 302. As in FIG. 2d, a new window has been defined associated with the window corresponding to the third pressure region 202, where the new window includes the third window assessment.

In this example, as with the example of FIG. 2d, the processing steps include determining whether there are any sharp peaks in the third window extension 213. In addition, the processing steps include performing a cross-correlation between pressure data and acceleration data in the third window extension 213. In this example, the cross-correlation steps are based on the same properties mentioned above. For this example, based on the cross-correlation assessment, it is determined that there is a correlation between the pressure data in the window extension and the acceleration data in the window extension. As can be seen, the sharp increase in acceleration is shortly followed a sharp increase in pressure. It is determined that the two are correlated, and it is determined that the presence of the sharp peak in pressure before the window corresponding to the third pressure region 202 is related to the acceleration, rather than a property of the body tissue 30 responding to the pressure. The third window extension 213 is then deemed to have not satisfied the third threshold criterion of including a sharp peak prior to the window corresponding to the third pressure region 202. As such, the determining the status of the body tissue 30 is determined based on the windows which satisfied the third threshold criterion. In this case, that is just the window corresponding to the second acceleration region 302.

These example processing steps may further facilitate processing of sensor data to identify patterns based on which the status of the body tissue 30 may be determined. These steps may also enable the removal of outliers in the data, or abnormalities in the data, which are less linked to the status of the body tissue 30. This may facilitate a reduction in the likelihood of false-positives, and/or false-negatives, where the determined status of the body tissue is incorrect (e.g. which results in unsuitable body tissue being used in a transplant and/or not using suitable body tissue in the transplant). In other words, embodiments of the present disclosure may provide more reliable monitoring of body tissue 30. The processing of data may also provide traceability such that the processing steps may be updated based on observed data, e.g. to account for instances where a determined status of the body tissue as deemed to be incorrect.

It will be appreciated in the context of the present disclosure that the exemplary system and methods described above may enable improved storage and preservation of body tissue 30, such as due to improved systems and methods for monitoring body tissue 30. However, it will also be appreciated that the system and methods described relate to specific examples. These examples are not to be considered limiting. Features described in these example are not necessarily essential, and systems and methods of the present disclosure may be provided without such features. Likewise, additional and/or alternative features may be provided. The following description is of some of these additional features and alternative arrangements.

Body Tissue Preservation System

Body tissue preservation systems of the present disclosure may include a container unit 10 and a base unit 60. The container unit 10 may be configured as an insert for the base unit 60. The container unit 10 may be sized and/or shaped according to a standard to facilitate insertion into the base unit 60. Each container unit 10 may be specific to a type of body tissue it is intended to be used with. For example, the container unit 10 may be of a selected size to receive its intended body tissue and/or the container may have a number of inlets and outlets depending on the intended body tissue (e.g. to provide preservation fluid to the correct number of lumens within the body tissue 30). The container unit 10 may comprise a restraint to secure a body tissue 30 within the body tissue receiving portion 12 of the container unit 10 (e.g. to inhibit movement of the body tissue 30 during transport).

The body tissue preservation system 100 may be for use for storage and/or transport of body tissue 30. The body tissue 30 may comprise an organ. For example, the system 100 may be arranged for storage and/or transport of organs to be used for organ transplants. The system 100 may be arranged to store and preserve the organ for an amount of time from that organ being harvested to the organ being ready for transplant into a patient. Container units may be organ specific. Each container unit 10 may comprise one or more indicia to indicate which type of organ that container unit 10 is for (e.g. they may be colour-coded).

Storage and/or preservation of body tissue 30 in the system 100 may comprise storing the body tissue 30 for a selected time period (e.g. associated with an amount of time between that body tissue 30 being harvested and being ready for that tissue to be transplanted into a patient). Storing the body tissue 30 for this selected time period may comprise controlling one or more properties of the body tissue 30 and/or its surrounding environment for preservation of the body tissue 30. Preserving the body tissue 30 may comprise retaining the body tissue 30 in a suitable condition for implant into a patient (e.g. after the selected time period has elapsed). For example, preserving the body tissue 30 may comprise inhibiting damage to the tissue after the tissue has been removed from a patient, and prior to that tissue being re-inserted to a patient.

The storage and/or preservation system 100 may comprise a body tissue 30 (e.g. organ) preservation system 100, such as an extracorporeal tissue storage and/or preservation system 100 (e.g. an ex vivo/ex situ system 100). The system 100 may be configured to deliver one or more fluids to body tissue 30 carried in the container unit 10. These fluids may comprise preservation fluids, and may be passed through one or more lumens of the body tissue 30 to facilitate preservation of the body tissue 30. The system 100 may be configured for gaseous and/or liquid perfusion of the body tissue 30 in the container unit 10. For example, the system 100 may be configured for one or more of: (i) normothermic liquid perfusion, (ii) hypothermic liquid perfusion, and (iii) persufflation, of the body tissue 30 in the container unit 10.

The storage and/or preservation system 100 may comprise a body tissue persufflation system 100. The body tissue persufflation system 100 may be configured to deliver one or more persufflation fluids to lumens of the body tissue 30. Persufflation fluids may be delivered in either a retrograde or anterograde manner. For anterograde persufflation, persufflation fluid (e.g. a persufflation gas) is delivered into the body tissue 30 through one or more arteries, and is taken out of the body tissue 30 from one or more veins (e.g. the flow of persufflation fluid enters through the artery and is drained through the vein). For retrograde persufflation, holes are pricked in the body tissue 30, and a persufflation fluid is delivered to the veins of the body tissue 30, and exits through the pricked holes. The persufflation fluid may comprise any suitable persufflation fluid configured to deliver oxygen to the body tissue 30, e.g. a persufflation gas with an oxygen level high enough to sustain the body tissue 30.

The container unit 10 may be a disposable (e.g. to prevent contamination of a later body tissue by an earlier body tissue carried by the container unit 10). The container unit 10 may comprise one or more base unit connectors to facilitate connection of the container unit 10 to the base unit 60. For example, the container unit 10 may comprise one or more flanges arranged to mate with a corresponding component of the base unit 60. The container unit 10 may store a pool of preservation fluid 34 in the body tissue receiving portion 12, e.g. so that a body tissue 30 stored in the container unit 10 is at least partially submerged in the preservation fluid 34.

The base unit 60 may comprise a source of preservation fluid, such as a canister of persufflation gas. The base unit 60 may be configured to be connected to the container unit 10 to supply this preservation fluid to the body tissue 30 in the container unit 10, such as to provide a fluid flow path for preservation fluid to arteries and/or veins of the body tissue 30. The base unit 60 may be configured to control delivery of this preservation fluid to the body tissue 30. The base unit 60 may also be configured to regulate a temperature/pressure of the environment of the body tissue 30 in the container unit 10. For example, a heater may be provided which is operable to raise the heat (and/or pressure) in the container unit 10, and/or a cooling device may be provided to reduce heat (and/or pressure) in the container unit 10.

Systems disclosed herein may include one or more sensors. Although examples described above include two sensors, this is not to be considered limiting. The system 100 may have one sensor, or it may have more than two. It is to be appreciated in the context of the present disclosure that the location and type of sensors used is not to be considered limiting. For example, any suitable sensor may be used, such as a temperature sensor, a pressure sensor, a vibration sensor, a humidity sensor, an oxygen concentration sensor, a flow meter, and/or a turbidity sensor. Suitable sensors may be configured to provide an indication of at least one property of the storage and/or preservation of body tissue 30. For example, sensors may provide an indication of a property which may be indicative of a status of the body tissue 30 being stored and preserved.

The container unit 10 may include one or more sensors. The sensors in the container unit 10 may be connected (e.g. wirelessly or by wired connection) to the controller 80. The sensors may be configured to enable the controller 80 to obtain an indication of one or measurements based on which the status of the body tissue 30 may be determined. For example, the container unit 10 may comprise at least one of: (i) a temperature sensor for the body tissue 30 and/or the environment surrounding the body tissue 30, (ii) a pressure sensor for the body tissue 30 (e.g. one of its lumens) and/or the environment surrounding the body tissue 30, (iii) a humidity sensor for the environment surrounding the body tissue 30, and (iv) an oxygen sensor for the environment surrounding the body tissue 30. The container unit 10 may comprise a movement sensor configured to sense one or more properties of movement of the container unit 10, such as an accelerometer. The container unit 10 may include a sensor and be configured to transmit data from that sensor to a controller 80 which is configured to monitor the data from that sensor as described herein.

Any suitable location for the one or more sensors may be used, such as within the first fluid store 72, between the first fluid store 72 and the fluid outlet 70 of the base unit 60, in the inlet 20 of the container unit 10, in the first channel 22, the second channel 24, the outlet 26 of the container unit 10, the fluid inlet 76 of the base unit 60, between the fluid inlet 76 and the second fluid store 74 and/or in the second fluid store 74. The sensor may be located in the container unit environment, such as in a surface of, or inside, the body tissue receiving portion 12. The location and/or type of sensor is not to be considered limiting. The arrangement may be configured to enable one or more operational parameters of the system 100 to be monitored to enable some feedback control of that system 100.

Sensors may be configured to provide measurement data indicative of the body tissue 30 and/or the environment surrounding the body tissue 30. This may include data indicative of movement of the body tissue 30 (within the system 100 as a whole). It may include physical properties of the body tissue 30 itself. Sensors may provide measurement values on a semi-regular basis. Sensors may provide a stream of measurement values. The sensors may be configured to output data which may enable the processing steps disclosed herein to be performed, e.g. data in which windows may be defined, such as data with sufficient granularity/resolution. The sensor may be configured to provide an ordered output of measurement values, such as a time-ordered output. The interval between measurements may be selected to enable micro and macro patterns to be identified within the measurement values. Sensor data may comprise data from one or more sensors. Sensor data may comprise a combination of data obtained from each of a plurality of sensors. Each sensor may output discrete or continuous data, such as a series of measurement values. The series of measurement values may be provided in a time-ordered series.

Examples of the present disclosure may provide a body tissue monitor. The body tissue monitor may comprise one or more sensors, and a controller 80 configured to perform the processing steps disclosed herein. For example, the body tissue monitor may be configured to monitor a body tissue and to determine a status of the body tissue based on sensor data for that body tissue. The sensor data may comprise one or more measurement values for a property of the body tissue and/or the environment surrounding it.

In examples described herein, the controller 80 is shown in a base unit 60. However, it is to be appreciated that this arrangement is not to be considered limiting. The controller 80 may be provided by any suitable component. The controller 80 may be connectable to one or more sensors to obtain data therefrom. This connection may be wired or wireless. For example, each sensor may be connected to a communications interface configured to enable transmission of data to the controller 80. The controller 80 may be provided in the container unit 10. The controller 80 may be a cloud-based service to which the sensors connect over a network and/or the controller 80 may be provided by a user device such as a mobile communications apparatus, e.g. a smart phone.

Automatic Control

Examples described herein relate to storage and preservation of body tissue 30, where the body tissue 30 is monitored to enable a status of the body tissue may be determined based on sensor data obtained for the body tissue 30. The monitoring of the body tissue 30 and determining its status may be used in an automatic control system for the storage and/or preservation of the body tissue 30. Based on the obtained sensor data, and the processing thereof performed by the controller 80, the automatic control system may control one or more operational parameters of the storage and/or preservation of the body tissue 30.

The controller 80 may be configured to control one or more operational parameters of the storage and/or preservation of the body tissue 30 in the container unit 10. The system 100 may include an adjuster which is operable to adjust a property of the body tissue 30 and/or the environment surrounding the body tissue 30. For example, the adjuster may be configured to adjust a property of the environment such as temperature, e.g. it may be a heater/cooler. The adjuster may be configured to control a property of preservation fluid delivered to the body tissue 30. The adjuster may be configured to control a pressure and/or flow rate of preservation fluid delivered to the body tissue 30. For example, the system 100 may include one or more controllable valves, such as in the base unit 60, which are operable to control flow characteristics of preservation fluid to the body tissue 30. Each controllable valve may be operated in a number of different states which permit different amounts of fluid to flow through to the body tissue 30. The controller 80 may be configured to control the operational state of the controllable valve.

Controlling an operational parameter may comprise controlling a parameter associated with fluids supplied to the body tissue 30 and/or a parameter for the environment of the body tissue 30 in the container unit 10. Controlling a parameter of the environment surrounding the body tissue 30 may comprise controlling one or more of: a temperature, a pressure, an oxygen concentration and/or a humidity of the environment of the body tissue 30, e.g. within the body tissue receiving portion 12 of the container unit 10. Controlling a parameter associated with a fluid supplied to the body tissue 30 may comprise controlling one or more of: a pressure of fluid supplied, a temperature of fluid supplied, a flow rate of fluid supplied, a number of active lines for the supply of fluid (e.g. a number of first channels to provide fluid to the body tissue 30), which particular fluid is supplied, and/or through which channels the fluid is to be supplied to the body tissue 30.

To provide automatic control, the controller 80 may be configured to control one or more operational parameters of the storage and/or preservation of body tissue 30. The controller 80 may control the at least one operational parameter based on the processing steps disclosed herein. For example, in the event that the controller 80 determines that a window of sensor data satisfies a relevant second threshold criterion, the controller 80 may adjust at least one parameter associated with the storage and/or preservation of the body tissue 30. For example, the controller 80 may control at least one operational parameter as a corrective measure in an attempt to control a property of the storage and preservation of the body tissue 30, e.g. as a corrective measure to try to correct for the second threshold criterion being satisfied.

The automatic control system may comprise detecting a window of sensor data satisfies a second threshold criterion and controlling operation of a component of the system 100 which may adjust the property associated with that window of sensor data. For example, where the second threshold criterion being satisfied for a window indicates that the fluid pressure has been sustained at a value which is too high, the automatic control may comprise adjusting a component, such as a controllable valve, which influences the pressure, e.g. to reduce the pressure. The automatic control system may provide a feedback loop to enable corrective measures to be initiated based on the processing steps disclosed herein indicating that a significant event has occurred (e.g. a second threshold criterion has been satisfied).

Each sensor may provide an indication of a property of the body tissue 30 and/or the environment surrounding the body tissue 30. Each sensor may have a corresponding adjuster which is operable to adjust that property. The automatic control may comprise using the corresponding adjuster to the sensor which has provided data indicating that a second threshold criterion has been satisfied. That corresponding adjuster may be controlled based on that second threshold criterion, e.g. to adjust the relevant property accordingly. Operational data for one or more such adjusters may be used when processing sensor data (e.g. to facilitate further cross-correlation between measurement values and adjuster operation, in case measurement data is indicative of adjuster operation rather than a more significant event).

In terms of automatic control, controlling an operational parameter of the storage and/or preservation of body tissue 30 may comprise controlling an output to the display screen 88 and/or restricting inputs from a user of the system 100. For example, a number of operational parameters may be displayed to the user using the display screen 88. Displayed parameters may include alerts that a significant event has occurred. A user of the system 100 may input data through the screen which controls one or more operational parameters of the system 100. The controller 80 may be configured to inhibit input data from a user which are determined to be contrary to any significant events which have occurred. For example, if the user tries to input instructions to increase pressure but a second threshold criterion associated with excessive pressure has been registered (or is currently occurring), the controller 80 may issue an alert and/or prevent this input data from being applied.

Examples described herein relate to the delivery of preservation fluid to the body tissue 30. However, it is to be appreciated in the context of the present disclosure that this is not to be considered limiting. For example, instead the ambient conditions in the environment of the body tissue 30 may be controlled, such as to control a temperature and/or pressure. This may occur without delivery of a preservation fluid.

It is to be appreciated in the context of the present disclosure that the processing of sensor data may be performed on-the-fly by the controller 80. That is, the controller 80 receives sensor data from the one or more sensors and processes this sensor (as described above). The sensor data may be continually being processed as the controller 80 receives it. At each instance where data is received which provides indication of a significant event (e.g. a threshold criterion is satisfied), the controller 80 may provide an output signal, such as to provide an alert or for automatic control. The controller 80 may be configured to process historical data, such as to receive historic sensor data from at least one sensor and to process this data as described herein. For example, a combination of retrospective and on-the-fly data processing may be provided, e.g. the controller 80 may store a buffer of recent sensor data, and the controller 80 may periodically process the sensor data in the buffer.

Output

Examples of the present disclosure described herein may provide a status output signal based on a determined status of the body tissue 30. It will be appreciated in the context of the present disclosure that the specific type of data output signal is not to be considered limiting. The status output signal may enable automatic control, as described above. For example, the status output signal may comprise a signal configured to control at least operational parameter of the storage and preservation of body tissue 30, e.g. based on the determined status of the body tissue 30.

The status output signal may be configured to provide an indication of the determined status of the body tissue 30, such as to be displayed on the display screen 88. The output signal may be configured to enable a user to select the level of detail with which they wish to view the sensor data and/or the determined status of the body tissue 30. The output signal may comprise a number of links which enable a user to select which level of detail they wish to use. The output signal may provide a layered output in the sense that the amount of data available to the user (e.g the granularity of the data available to the user) may be selected by the user by selecting the relevant data link.

For example, the status output signal may at a top layer provide an indication of whether or not the organ is determined to be viable for transplant. A subsequent layer may provide a probabilistic value of the likelihood that the body tissue 30 is viable for transplant and/or an indication of deterioration of the body tissue 30 during storage. A subsequent layer may provide an indication of the most significant events, such as those which satisfied the highest level threshold criterion (e.g. second or third). A subsequent layer may provide an indication of the trigger events in the sensor data. A final layer may provide the sensor data in its entirety. As will be appreciated, in each subsequent layer more data may be provided. The links in the data may facilitate switching between different layers.

This arrangement of the data output may enable a surgeon to receive a determined indication of the status of the body tissue 30. The surgeon may use the different links/levels to view the relevant portions of the sensor data based on which the status of the body tissue 30 has been determined. The links may be to the different windows in the data (e.g. the different time windows associated with satisfying their respective threshold criteria). Using a particular link may provide the user (e.g. the surgeon) with the data corresponding to that link (e.g. the relevant subset of the data that satisfied a threshold criterion). This may enable the surgeon to verify the determined status of the body tissue 30. In particular, this arrangement may enable the surgeon to minimise their time reviewing the sensor data for transport, as the amount of sensor data they review may be reduced so that only the more significant events are shown. This arrangement of data may also facilitate direct verifiability, auditability and/or traceability in the determined status of the body tissue 30. For example, it may be directly identified based on the sensor data, and the identified significant events, why the status of the body tissue 30 was determined. That way, in the event of any issue with a transplanted organ, it may be established what went wrong in the data processing, and/or a surgeon may be able to ascertain why the status of the body tissue 30 was determined in the way it was. This direct verifiability and traceability may find particular utility for organ transplants.

Determining Status of Body Tissue

Examples disclosed herein are configured to determine a status of a body tissue 30 based on the obtained sensor data. Sensor data may be assessed on both a ‘micro’ and a ‘macro’ level. That is, instantaneous measurement values may be assessed (micro level) and a plurality of measurement values may be assessed together (macro level). Based on both of these levels (micro/macro) the status of the body tissue 30 may be determined. It is to be appreciated in the context of the present disclosure that the specific selection of these levels is not to be considered limiting. In this regard, two types of region of sensor data may be defined. The first type of region may be referred to as an ‘event’. An event may be considered to be a micro scale occurrence in the sensor data. The second type of region may be referred to as an ‘activity’. An activity may be considered to be made up of one or more events. Where more levels are used (e.g. third threshold criterion is to be applied), the third level may be made up of one or more activities.

The controller 80 is configured to detect trigger events. Trigger events may provide an indication of a significant event occurring. It is to be appreciated in the context of the present disclosure that the precise nature of trigger events described herein is not to be considered limiting. To define a trigger event, a first threshold criterion is provided which, if satisfied, indicates that a trigger event has occurred. Any suitable first threshold criterion may be used. The first threshold criterion may be selected based on empirical data or theoretical data. The first threshold criterion may be selected based on data which suggests that an event has occurred/is occurring/is about to occur which may influence the status of the body tissue 30. The first threshold criterion may be selected so that, in the event that the first threshold criterion is satisfied, this indicates that processing of additional sensor data associated with that trigger event may enable an improved determination of the status of the body tissue 30.

The sensor data may comprise measurements from one or more sensor. The first threshold criterion may be based on expected/target values for a parameter measured by a sensor, and/or threshold values for a parameter measured by a sensor. The first threshold criterion may be selected so that it is satisfied in the event that the measurement value for the parameter is far from the expected/target value and/or exceeds the threshold value (e.g. is outside a range between threshold values). The first threshold criterion may provide an indication that the measurement value has entered into a region where review of the measurement value in that region may be significant to the determination of the status of the body tissue 30.

The first threshold criterion may not be based on an absolute value for a measurement. For example, the first threshold criterion may be based on a property of how the measurement value has changed, such as a gradient, a shape and/or a pattern of the measurement values within the sensor data. For example, the first threshold criterion may be satisfied in the event that the gradient of measurement values (e.g. change between subsequent values exceeds a threshold value). The first threshold criterion may not be assessed based on one instantaneous measurement in the sensor data. For example, the first threshold criterion may be assessed on the basis of a plurality of measurements in the sensor data. This assessment may be based on each of the measurements (such as a series of measurements conforming to a certain pattern/outside a threshold range) or it may be based on a processed form of those measurements (such as an average over a selected time period).

In the event that a trigger event is detected, the controller 80 is configured to select a window of the sensor data associated with the trigger event. Sensor data corresponding to this window may then be assessed based on the second threshold criterion. In this regard, assessment of whether the second threshold criterion is satisfied may comprise an assessment of whether an activity satisfies a threshold criterion (whereas satisfying the first threshold criterion may comprise an assessment of whether an event satisfies a threshold criterion). The window may be selected to encompass more sensor data than that used in the assessment of whether the first threshold is satisfied.

Selecting the window of data may comprise selecting data for measurements before and/or after the trigger event. The window may be selected to encompass the sensor data giving rise to the trigger event. The selected window may encompass more data based on which larger scale patterns may be identified (e.g. on a scale larger than those identified when detecting the trigger event). The window of data may be selected to encompass all data which satisfies a selected condition, e.g. all data proximal to the data giving rise to the trigger event which satisfies the selected condition (e.g. a relevant threshold criterion). The selected window may comprise data from another sensor. For example, the selected window may include data from all sensors, or it may include data from only one, or a selected number, of sensors. For example, the sensors may be selected based on relationships between the parameters the sensors measure (such as to encompass data which may be relevant, but to avoid unnecessary data). Selecting the window may comprise selecting a plurality of windows (e.g. each for respective sensor data), and selecting the window may be based on cross-correlation of sensor data for the assessment of satisfying threshold criteria. For example, windows may be selected to enable application of systems and methods of cross-correlation as disclosed herein.

It is to be appreciated in the context of the present disclosure that the window need not be a time window. For example, selecting the window may comprise selecting a number of measurement values either side of the trigger event. The number of measurement values may be independent of time (e.g. the sensor may provide measurement values at random). For example, selecting the window may comprise selecting based on a property of the system 100, e.g. based on location within a cyclical motion. The window may be selected to enable larger scale patterns in the sensor data to be identified. The selected window may provide more sensor data to be assessed (against the second threshold criterion).

Based on the selected window, the relevant subset of sensor data is identified for that window. The subset of data may comprise all data in that window (e.g. across multiple sensors). The subset of data may comprise some of the data in that window (e.g. measurements from some, but not all, of the sensors). The data identified may be selected based on the second threshold criterion.

The second threshold criterion may be assessed in a similar manner to that of the first threshold criterion. However, the assessment of the second threshold may be based on the data in the selected window, and the selected window may encompass more data than that against which the first threshold criterion was assessed. The assessment of the second threshold criterion may comprise any suitable assessment. The second threshold criterion may be different to the first threshold criterion. Satisfying the second threshold criterion may comprise an indication of a larger scale trend or property being apparent within the sensor data.

The second threshold criterion may be selected to provide a more significant indication as to the status of the body tissue 30. For example, the second threshold criterion being satisfied may provide a more statistically significant indication as to the status of the body tissue 30 than the first threshold criterion being satisfied. This indication could be either positive or negative as to the status of the body tissue 30, e.g. that it is healthy or that it is not, such as that minimal deterioration has occurred or significant deterioration has occurred. In the event that at least one second threshold criterion is satisfied, the determined status of the body tissue 30 may be considered to be more reliable than if no second threshold criterions have been satisfied. The second threshold criterion may be associated with a response in the sensor data known to correspond to a particular indication of the status of the body tissue 30. For example, the second threshold criterion may be selected as an indicator that the sensor data corresponds to a known status for the body tissue 30, where that indicator is more closely-linked to the body status than for a first threshold criterion.

The status of the body tissue 30 may be determined based on one or more subsets in the sensor data which satisfied the second threshold criterion. It will be appreciated in the context of the present disclosure that the status of the body tissue 30 may be determined based on the sensor data, and what threshold criteria are satisfied within the sensor data. For example, where satisfying the second threshold criterion provides a statistically significant indication as to the status of the body tissue 30, the status of the body tissue 30 may be determined on the basis of the number of instances within the sensor data where a second threshold criterion is satisfied. For example, the controller 80 may determine a score based on the sensor data, and the status of the body tissue 30 is based on the determined score. The score may be based on a number of second threshold criteria satisfied. The score may be based on the extent to which each second threshold criteria was satisfied, e.g. if the subset clearly or marginally satisfied the second threshold criterion.

The controller 80 may be configured to determine the status of the body tissue 30 based on a weighted combination of the sensor. The regions within the sensor data which are deemed to be those most indicative of the status of the body tissue 30 may be assigned a greater weighting (e.g. these may be the windows which satisfied the second threshold criterion). The regions within the sensor data which are deemed to be at least partially indicative of the status of the body tissue 30 may be assigned a medium weighting (e.g. these may be regions in the sensor data where the first threshold criterion was satisfied, but where the second threshold criterion for the corresponding window was not satisfied). The regions within the sensor data which are less indicative of the status of the body tissue 30 may be assigned no, or a small, weighting (e.g. these may be regions in which the first threshold criterion was not satisfied. Any suitable combination and/or processing of the sensor may be provided which enables an indication of the status of the body tissue 30 to be determined. This processing of sensor data may be based on historical data for body tissue monitoring where the status of the body tissue being monitored is known.

Determining the status of the body tissue may comprise following an algorithmic approach. A series of operators may be defined for processing the sensor data. For example, for each of the trigger events, their corresponding identified subsets of data may be processed according to one or more operators. Each operator may comprise a function for processing the data to provide an output which may enable the status of the body tissue 30 to be determined. Each of the subsets of the data which satisfied the second threshold criterion may be processed according to one or more operators. The output from these operators may provide the status of the body tissue 30, e.g. where an output from an operator indicates a status of the body tissue 30 above a threshold degree of significance, the body tissue status may be determined based on this operator.

The sensor data may comprise data from one or more sensors. The determination of the status of the body tissue 30 may be based on data from multiple sensors. Data from each sensor may be processed to determine an indication of the status of the body tissue 30, e.g. as described above. The status of the body tissue 30 may be determined based on the data from multiple sensors, e.g. not based on data from one sensor alone. The status of the body tissue 30 may be determined taking data from multiple sensors into account at the same time. Data from different sensors may be cross-correlated, e.g. so that both sets of data and/or the relationship between them, are considered when determining the status of the body tissue 30.

It is to be appreciated in the context of the present disclosure that any suitable process for cross-correlation of data may be used. Two sets of data may be compared to identify any correlation therebetween. For example, a score may be determined for the correlation between any events/activities in two sets of data, and if the score is above a threshold value, then it is determined that the two events/activities are related to one another. Cross-correlation may be performed based on any relevant region of each set of data. For example, a window in a first set of data may be compared to a corresponding window in a second set of data. As another example, a window in a first set of data may only be compared to the second data set if there is also a window defined therein (e.g. which satisfied the second threshold criterion). Whether sensor data satisfies the first and/or second threshold criterion may be based on multiple sets of data, and a correlation therebetween. For example, the threshold criteria may apply to multiple sources of data such that a criterion may be satisfied based on both sets of data when either data set in isolation would not satisfy the criterion. Likewise, a criterion may only be satisfied when both sets satisfy the criterion (and/or so does their combination/correlation), even if in isolation those data sets may satisfy the threshold criterion.

It is to be appreciated in the context of the present disclosure that although examples described herein relate to a body tissue preservation system 100, such as a persufflation system, this is not to be considered limiting. Aspects of the present disclosure may relate to a body tissue monitor for monitoring a body tissue to determine a status of the body tissue. For example, any suitable body tissue may be monitored. Any suitable sensor may be provided for this monitoring to enable processing of the sensor data as described herein. For example, body tissue such as organs (heart/lungs etc.) and their functionality may be monitored, such as using an echocardiogram or other suitable arrangement, e.g. to monitor the status of a patient and the status of one or more of their body tissues, such as their organs. It will be appreciated in the context of the present disclosure that any relevant body tissue based on which suitable sensor data may be obtained may be monitored using processes described herein for monitoring body tissue.

In examples described herein, events which satisfy threshold criteria may be deemed to be events which have an impact on the body tissue, e.g. so that the status of that body tissue may be determined based on those events. It is to be appreciated in the context of the present disclosure that such events may typically indicate harm to the body tissue (e.g. as opposed to that the tissue is healthy). For example, a large and sustained spike in pressure may indicate that the likelihood of that body tissue being suitable for transplant is very low. However, it will be appreciated that this is not limiting. For example, the absence of any abnormalities (e.g. there being no regions satisfying threshold criteria) may be considered to provide an indication that the body tissue is healthy. The absence of abnormalities may also provide an indication that one or more of the sensors are not working properly. The controller 80 may be configured to identify this based on cross-correlation between sensors and/or by detecting insufficient variation in the sensor data (e.g. in which case a different threshold criterion may be satisfied). In other examples, patterns and/or values indicative of healthy tissue may be identified.

Alternatives, Variants and/or Additional Features

In examples described herein, fluid flow paths between the base unit 60 and the container unit 10 are described. It is to be appreciated in the context of the present disclosure that the flow paths discussed are merely exemplary. For example, the base unit 60 may contain a source of preservation fluid, and one or more flow paths may be defined to enable that preservation fluid to be delivered to the body tissue. The flow path may go through an inlet 20 in the container unit 10, or tubes may be provided which may pass over the sides of the container unit 10 and into the body tissue receiving portion 12. A plurality of fluid flow paths may be provided to enable preservation fluid to be delivered to the body tissue in the container unit 10. For example, the container unit 10 may be arranged to enable a plurality of first channels to be connected to the body tissue for the delivery of preservation fluid. The container unit 10 may also not have an outlet 26. For example, where a gas is to be pumped into the body tissue, the gas may pass from the body tissue into the surrounding environment, e.g. and then out through a vent/filter to the atmosphere.

The container unit 10 may include one or more fluid processing elements. The fluid processing elements may include a passageway sized and/or shaped to provide some processing of the fluid therein, such as to reduce bubble size, humidify and/or cool the fluid therein. These may be used to process incoming preservation fluid which is to be delivered to the body tissue. For example, the incoming preservation fluid may be a persufflation gas, and the fluid processing elements may process this fluid between being received at the inlet 20 of the container unit 10 and passing through the first channel 22 into the body tissue.

Although examples have been described which include a flange 14, a flange 14 may not be included. For example, the base unit 60 may comprise an attachment for retaining the container unit 10 within the container unit receiving portion 62 of the base unit 60, such as a strap, hook or other attachment means to secure the container unit 10 in place. The container unit 10 may include a suitable attachment to ensure it is retained in the base unit 60. The container unit 10 may be provided without any flange 14 or attachment.

Examples described herein include a first and second fluid storage tank. However, it is to be appreciated that one or both of these may not be included. For example, the base unit 60 may be configured to generate preservation fluid in situ. The first fluid store 72 may be a gas canister storing persufflation fluid. The second fluid store 74 may not be included, such as where the preservation fluid may be expelled from the container unit 10 into the atmosphere, or where the fluid inlet 76 of the base unit 60 is connected to an expulsion portal for expelling/draining used preservation fluids. The preservation fluid may be recycled (e.g. cleaned and process to ensure the oxygen concentration is within a threshold level) within the base unit 60, e.g. to enable this preservation fluid to be re-delivered to the body tissue.

Some examples described herein include a controllable valve, which may be used to control the at least one operational parameter of the storage and/or preservation of the body tissue in the container unit 10. However, it is to be appreciated in the context of the present disclosure that any suitable component may be provided to facilitate control of at least one operational parameter of the storage and/or preservation of body tissue. For example, a heater/cooler may be include to regulate the temperature of preservation fluid and/or to regulate ambient temperature in the body tissue's environment. Other regulators may include an oxygen supply to regulate an oxygen concentration of preservation fluid, a selectable valve to control fluid flow from more than one fluid source (e.g. to select which fluid source(s) to use).

It will be appreciated from the discussion above that the examples shown in the figures are merely exemplary, and include features which may be generalised, removed or replaced as described herein and as set out in the claims. With reference to the drawings in general, it will be appreciated that schematic functional block diagrams are used to indicate functionality of systems and apparatus described herein. In addition the processing functionality may also be provided by devices which are supported by an electronic device. It will be appreciated however that the functionality need not be divided in this way, and should not be taken to imply any particular structure of hardware other than that described and claimed below. The function of one or more of the elements shown in the drawings may be further subdivided, and/or distributed throughout apparatus of the disclosure. In some examples the function of one or more elements shown in the drawings may be integrated into a single functional unit.

As will be appreciated by the skilled reader in the context of the present disclosure, each of the examples described herein may be implemented in a variety of different ways. Any feature of any aspects of the disclosure may be combined with any of the other aspects of the disclosure. For example method aspects may be combined with apparatus aspects, and features described with reference to the operation of particular elements of apparatus may be provided in methods which do not use those particular types of apparatus. In addition, each of the features of each of the examples is intended to be separable from the features which it is described in combination with, unless it is expressly stated that some other feature is essential to its operation. Each of these separable features may of course be combined with any of the other features of the examples in which it is described, or with any of the other features or combination of features of any of the other examples described herein. Furthermore, equivalents and modifications not described above may also be employed without departing from the invention.

Certain features of the methods described herein may be implemented in hardware, and one or more functions of the apparatus may be implemented in method steps. It will also be appreciated in the context of the present disclosure that the methods described herein need not be performed in the order in which they are described, nor necessarily in the order in which they are depicted in the drawings. Accordingly, aspects of the disclosure which are described with reference to products or apparatus are also intended to be implemented as methods and vice versa. The methods described herein may be implemented by a controller, such as in computer programs, or in hardware or in any combination thereof. Computer programs include software, middleware, firmware, and any combination thereof. Such programs may be provided as signals or network messages and may be recorded on computer readable media such as tangible computer readable media which may store the computer programs in non-transitory form. Hardware includes computers, handheld devices, programmable processors, general purpose processors, application specific integrated circuits (ASICs), field programmable gate arrays (FPGAs), and arrays of logic gates.

In some examples, one or more memory elements can store data and/or program instructions used to implement the operations described herein. Embodiments of the disclosure provide tangible, non-transitory storage media comprising program instructions operable to program a processor to perform any one or more of the methods described and/or claimed herein and/or to provide data processing apparatus as described and/or claimed herein.

Other examples and variations of the disclosure will be apparent to the skilled addressee in the context of the present disclosure.

1. A body tissue preservation system for storage and preservation of body tissue, wherein the body tissue preservation system comprises a body tissue monitor for monitoring a body tissue to determine a status of the body tissue, wherein the body tissue monitor comprises:
at least one sensor configured to obtain sensor data based on a plurality of measurements of the body tissue and/or the environment surrounding the body tissue; and
a controller arranged to receive the sensor data from the at least one sensor, wherein the controller is configured to:
detect one or more trigger events in the sensor data, wherein each trigger event comprises sensor data which satisfies a first threshold criterion;
select, for each of the one or more trigger events, a window of the sensor data associated with the trigger event;
identify, for each of the one or more windows, a subset of the sensor data corresponding to the selected window;
determine, for each of the one or more selected windows, whether the corresponding identified subset of sensor data satisfies a second threshold criterion;
determine a status of the body tissue based on identified subsets which satisfied the second threshold criterion; and
provide a status output signal based on the determined status of the body tissue.

2. The body tissue preservation system of claim 1, wherein the system is configured to store and preserve extracorporeal body tissue.

3. The body tissue preservation system of claim 1, wherein the system is configured to persufflate the body tissue in the container unit.

4. The body tissue preservation system of claim 1, wherein the body tissue is body tissue for a transplant, and the system is configured to monitor the body tissue during storage and/or transport; and
wherein the status output signal provides an indication of the viability of the body tissue for transplant.

5. The body tissue preservation system of claim 1, wherein the system comprises a container unit arranged to receive the body tissue; and
wherein the container unit comprises the at least one sensor.

6. The body tissue preservation system of claim 1, wherein the system includes at least one adjuster operable to adjust a property of the body tissue and/or the environment surrounding the body tissue.

7. The body tissue preservation system of claim 6, wherein the controller is configured to control operation of the adjuster to adjust the property of the body tissue and/or the environment surrounding the body tissue in the event that it determines that an identified subset of the sensor data satisfies the second threshold criterion.

8. The body tissue preservation system of claim 3, wherein the system includes at least one adjuster operable to adjust a property of the body tissue and/or the environment surrounding the body tissue, and wherein the controller is configured to control the adjuster to adjust at least one of: (i) a flow of persufflation fluid to the body tissue, and (ii) a pressure of persufflation fluid flowing through the body tissue.

9. The body tissue preservation system of claim 1, wherein selecting a window of the sensor data comprises selecting a time window of the sensor data.

10. The body tissue preservation system of claim 1, wherein the system comprises a plurality of sensors, each configured to obtain sensor data based on a plurality of measurements for the body tissue and/or the environment surrounding the body tissue.

11. The body tissue preservation system of claim 10, wherein the controller is configured to determine whether an identified subset of data from a first sensor satisfies the second threshold criterion based also on data from a second sensor.

12. The body tissue preservation system of claim 11, wherein in the event that the controller detects a trigger event in sensor data from the first sensor, the controller is configured to select a window associated with the trigger event in both the sensor data from the first sensor and the sensor data from the second sensor.

13. The body tissue preservation system of claim 1, wherein the controller is configured to detect that sensor data satisfies the first threshold criterion in the event that at least one of: (i) the sensor data is outside a selected range, (ii) a change in the sensor data is above a threshold amount, and (iii) a time threshold has been reached.

14. The body tissue preservation system of claim 1, wherein the controller is configured to determine that an identified subset of the sensor data satisfies a second threshold criterion in the event that at least one of: (i) the sensor data is outside a selected range for a threshold period of time within the selected time window, (ii) the sensor data changes more than a threshold amount within the selected time window, and (iii) one or more selected patterns are identified in the sensor data within the selected time window.

15. The body tissue preservation system of claim 1, wherein the controller is configured to output an alert in the event that it is determined that a corresponding identified subset of the sensor data satisfies the second threshold criterion.

16. The body tissue preservation system of claim 1, wherein the status output signal includes each identified subset of the sensor data which satisfied the second threshold criterion.

17. The body tissue preservation system of claim 16, wherein the status output signal includes the identified subsets of the sensor data.

18-22. (canceled)

23. A body tissue monitor for monitoring a body tissue to determine a status of the body tissue, wherein the body tissue monitor comprises:
at least one sensor configured to obtain sensor data based on a plurality of measurements of the body tissue and/or the environment surrounding the body tissue; and
a controller arranged to receive the sensor data from the at least one sensor, wherein the controller is configured to:
detect one or more trigger events in the sensor data, wherein each trigger event comprises sensor data which satisfies a first threshold criterion;
select, for each of the one or more trigger events, a time window of the sensor data associated with the trigger event;
identify, for each of the one or more time windows, a subset of the sensor data corresponding to the selected window;
determine, for each of the one or more selected time windows, whether the corresponding identified subset of sensor data satisfies a second threshold criterion;
determine a status of the body tissue based on identified subsets which satisfied the second threshold criterion; and
provide a status output signal based on the determined status of the body tissue.

24. A method of storing and preserving body tissue, wherein the method comprises:
obtaining sensor data defining a stream of measurements for the body tissue and/or the environment surrounding that body tissue;
detecting one or more trigger events in the sensor data, wherein each trigger event comprises sensor data which satisfies a first threshold criterion;
selecting, for each of the one or more trigger events, a window of the sensor data associated with the trigger event;
identifying, for each of the one or more windows, a subset of the sensor data corresponding to the selected window;
determining, for each of the one or more selected windows, whether the corresponding identified subset of the sensor data satisfies a second threshold criterion;
determining a status of the body tissue based on identified subsets which satisfied the second threshold criterion; and
providing a status output signal based on the determined status of the body tissue.

25. A computer program product comprising computer program instructions configured to program a controller to perform the method of claim 24.
